(19) United States
(12) Patent Application Publication
(10) Pub. No.: US 2007/0238745 A1
(43) Pub. Date: Oct. 11, 2007

(54) PI3K-Akt Pathway Inhibitors

(75) Inventors: Subhra Mohapatra, Tampa, FL (US); W. Jack Pledger, Odessa, FL (US)

Correspondence Address: SMITH HOPEN, PA, 180 PINE AVENUE NORTH, OLDSMAR, FL, 34677, US

(73) Assignee: UNIVERSITY OF SOUTH FLORIDA, Tampa, FL (US)

(21) Appl. No.: 11/733,001

(22) Filed: Apr. 9, 2007

Related U.S. Application Data
(60) Provisional application No. 60/744,448, filed on Apr. 7, 2006.

(51) Int. Cl. A61K 31/52; A61K 31/452

(52) U.S. Cl. 514/263.4; 514/320

(57) ABSTRACT

A treatment for cancer using a combination therapy including an inhibitor of the PI3K/Akt pathway in combination with roscovitine. It is shown that the combination of roscovitine and API-2 (Triciribine) or roscovitine and LY294002 induce the apoptosis of androgen-dependent (LNCaP) and androgen-independent (PC3) prostate cancer cells. Two important results have been observed. First, cells that respond to roscovitine alone (LNCaP) initiate apoptosis sooner when co-treated. Second, cells that do not respond to roscovitine alone (PC3) apoptose when co-treated, although with delayed kinetics. In the absence of roscovitine, AKT inhibitors had no effect on LNCaP or PC3 survival, and in both cell lines, the combined treatment activated the mitochondrial pathway of apoptosis. Importantly, normal epithelial cells (RPWE) remained viable in the presence of roscovitine and AKT inhibitors. Events elicited by roscovitine (down-regulation of XIAP) and AKT inhibitors (accumulation of Bim) in LNCaP and PC3 cells are identified. Additional data show that PC3 cells apoptose when treated with AKT inhibitors and depleted of either XIAP or Cdk9. Taken together, these important results lead to improved treatments for cancers, such as prostate cancer, through the combination therapies taught herein.

### CROSS REFERENCE TO RELATED APPLICATIONS

This application claims priority to currently pending U.S. Provisional Patent Application 60/744,448, entitled, “PI3K-Akt Pathway Inhibitors”, filed Apr. 7, 2006, the contents of which are herein incorporated by reference.

### STATEMENT OF GOVERNMENT INTEREST

This invention was made with Government support under Grant No. 93544-01 awarded by the National Cancer Institute and under Grant No. 04-NIR-11 awarded by NIR-Florida Department of Health. The Government has certain rights in the invention.

### FIELD OF INVENTION

This invention relates to cancer therapy. More specifically, this invention relates to combination therapy with roscovitine and an Akt inhibitor for the treatment of cancer.

### BACKGROUND OF THE INVENTION

Prostate cancer is the third leading cause of death among men in America, after lung cancer and colorectal cancer, and accounts for approximately one-third of cancers in men [Feldman, B. J. and D. Feldman, 2001]. It is estimated that more than 250,000 men will develop prostate cancer in 2007 and about 30,000 men will die from it. Though the majority (more than 75 percent) of cases occurs in men over age 65, many cases also occur in younger men, who sometimes have a more aggressive cancer. Prostate cancer mortality results from metastases to bones and lymph nodes and progression from androgen-dependent to androgen-independent disease. Although androgen deprivation has been found to be effective in treating androgen-dependent prostate cancer, no effective life-prolonging therapy is available for the aggressive, metastasizing and androgen independent prostate cancer. The later tumors are largely refractory to standard chemotherapy regimens, and most patients at this disease stage die within a few years. The need to identify agents that kill androgen-independent prostate cancer cells is of great importance. The present invention meets this crucial need by providing a combination treatment of roscovitine and API-2 that induces significant apoptosis of prostate cancer cells, irrespective of androgen dependence. Furthermore, we have identified key intracellular targets: Cdk9, XIAP, Bim and p53 that will provide framework for elucidation of mechanism of apoptosis.

### SUMMARY OF INVENTION

A treatment for cancer using a combination therapy including an inhibitor of the PI3K/Akt pathway in combination with a Cdk9 or XIAP inhibitor, such as roscovtine or flavopiridol.

In a first aspect the present invention provides a combination therapy including API-2 (Triciribine) and roscovitine. The combination therapy is particularly suited for the treatment of cancer such as prostate cancer. The combination is administered to a subject in need of such a combination in a therapeutically effective amount.

In a second aspect the present invention provides a combination therapy for the treatment of cancer comprising a therapeutically effective amount of roscovitine and one or more PI3K/Akt inhibitors. The one or more PI3K/Akt inhibitors can be of API-2 or LY (LY294002).

In a third aspect the present invention provides a combination therapy for the treatment of cancer comprising a therapeutically effective amount of a Cdk9 inhibitors and one or more PI3K/Akt inhibitors. The one or more PI3K/Akt inhibitors can be of API-2 or LY (LY294002). The Cdk9 inhibitor can be roscovitine or flavopiridol.

Also provided in the present invention are methods of treating cancer. In a first aspect of these methods there is provided a method of treating prostate cancer in a subject. The method includes the step or steps of administering roscovitine and one or more PI3K/Akt inhibitors in a therapeutically effective amount to a subject in need thereof. The method can further include the step of performing androgen ablation therapy. In certain embodiments the method is used to treat androgen-independent prostate cancer. The PI3K/Akt inhibitor can be utilized in the method increase Bim abundance. In certain embodiments the one or more Akt inhibitors can be API-2 or LY (LY294002).

In a second aspect of these methods there is provided a method of treating prostate cancer in a subject. The method includes the step or steps of administering administering in combination API-2 and one or more inhibitors of XIAP. In certain embodiments the XIAP inhibitor depletes Cdk-9. In certain embodiments the XIAP inhibitor is roscovitine.

In a third aspect of these methods there is provided a method of treating androgen-independent prostate cancer in a subject. The method includes the step or steps of administering roscovitine and API-2 in a therapeutically effective amount to a subject in need thereof.

In a fourth aspect of these methods there is provided a method of treating cancer in a subject including the step of administering roscovitine and one or more PI3K/Akt inhibitors in a therapeutically effective amount to a subject in need thereof. In certain embodiments the one of the one or more PI3K/AKT inhibitors can be API-2 or LY294002. Treated cancers can include prostate cancer, sarcoma and mantle cell lymphoma.

In a fourth aspect of these methods there is provided a method for treating a tumor or cancer in a mammal comprising (i) obtaining a biological sample from the tumor or cancer; (ii) determining whether the tumor or cancer overexpresses an Akt kinase, (iii) if the tumor or cancer overexpresses Akt kinase, treating the tumor or cancer with an effective amount of a combination therapy comprising API-2 and roscovitine. In certain embodiments the level of Akt kinase expression is determined by assaying the cancer for the presence of a phosphorylated Akt kinase. The treated mammal can be a human. Furthermore, the cancer can be prostate cancer. In certain embodiments the prostate cancer is androgen-independent prostate cancer. In alternative embodiments the prostate cancer treatment can include the step of performing androgen ablation therapy. In still further embodiments the step of determining whether the cancer expresses p53, wherein the expression of wt p53 correlates favorably with responsiveness to treatment with the combination therapy API-2 (Triciribine) and roscovitine.

### BRIEF DESCRIPTION OF THE DRAWINGS

For a fuller understanding of the invention, reference should be made to the following detailed description, taken in connection with the accompanying drawings, in which:

FIG. 1 is a series of Western blots illustrating the increased apoptosis of LNCaP cells treated with a combination of roscovitine and AKT inhibitors. In FIG. 1A LNCaP cells were cultured in presence of optimal dosages one of the inhibitors of PKA (H89), JNK (SP6), PKC (Bis), p38 (SB), PI3K (Wortmannin and LY294002), MEK (U0126) in presence or absence of roscovitine for 8 hrs. Cell lysates were analysed for PARP cleavage by Western blotting. In FIG. 1B LNCaP cells were treated with 10 uM LY294002 or 0.5 uM Wortmannin in presence or absence of roscovitine for 8 hrs. Lysates were analysed by Western blotting. In FIG. 1C LNCaP cells were treated with 10 uM LY and 25 uM roscovitine for indicated periods. Lysates were analysed by Western blotting. In FIG. 1D LNCaP cells were transfected with pcDNA3 (vector) or pcDNA3 encoding dn-AKT plasmid. Twenty-four hrs after transfection cells were treated with 15 uM roscovitine and incubated for 16 hrs. Lysates were analysed by Western blotting.

FIG. 2 is a pair of histograms showing combination treatment induces apoptosis of LNCaps but not normal prostate epithelial cells. In FIG. 2A combination with LY and roscovitine induced caspase-dependent apoptosis in LNCaP cells. Cells were cultured for 4 hrs with Ly and/or Roscovitine in presence or absence of pan, caspase-3 and caspase-9 inhibitors. Amounts of cytosolic histone-associated DNA fragments were determined by Cell Death ELISA. In FIG. 2A combination treatment did not induce apoptosis of RWPE cells. Cells were treated with LY and/or roscovitine for 20 hrs and amounts of cytosolic histone-associated DNA fragments were determined by Cell Death ELISA.

FIG. 3 is a series of graphs illustrating the effects of drugs on growth potential of prostate cancer cells. Cells were treated with various inhibitors for 72 hrs and cell growth was enumerated by trypan-blue exclusion.

FIG. 4 illustrates that a combination of roscovitine and AKT inhibitors suppresses colony formation. For FIGS. A-C cells were plated with LY or API-2 and roscovitine for 46 hrs (or indicated hours) and then 10⁴ viable cells from each treatment condition were re-plated in 100 mm dish in the presence of complete culture medium for additional 2 weeks. Medium was replaced twice. (FIG. 4A) Colony formation in PC3 cells exposed to drugs for 46 hrs. (FIG. 4B) Colony numbers for PC3 cells exposed to drugs for 24 or 46 hrs. (FIG. 4C) Colony numbers for PC3MM2 cells exposed to drugs for 46 hrs. (FIG. 4D) Morphological alterations in PC3 cells. PC3 cells were cultured for 24 and 48 hrs in presence or absence of indicated combination of drugs.

FIG. 5 illustrates caspase-dependent apoptosis of PC3 cells when co-treated with roscovitine and API-2. (FIG. 5A) Increased DNA Fragmentation observed when PC3 cells co-treated with roscovitine and AKT inhibitors. PC3 cells were treated with 20 mM LY, 20 mM API-2, 50 mM roscovitine or combination for 14 hr. Amounts of cytosolic histone associated DNA fragments were assessed using Cell Death ELISA assay (Roche) (FIG. 5B) PC3 cells were treated with indicated dosages of roscovitine and API-2 for 40 hrs. Tunel positive cells are shown. (FIG. 5C)PC3 cells were treated with 20 uM API-2, 25 uM roscovitine or both for 40 hrs. Cells were incubated for 60 mins with a cell permeable caspase-inhibitor (sulforhodamine-labeled fluoromethyl ketone) which binds to active caspases. Cells were incubated with Hoechest stain for 5 mins and examined using a fluorescent microscope. (FIG. 5D) PC3 cells were treated with indicated concentrations of roscovitine, API-2 or both in presence or absence of caspase inhibitors for 14 hrs. Amounts of cytosolic histone associated DNA fragments were measured.

FIG. 6 illustrates the combination treatment targets Akt, RNA-Pol II, XIAP and Bim in prostate cancer cells. (FIG. 6A) Early exposure to drug combination inhibits Akt and RNA-pol II activities and induce Bim. PC3 cells were exposed to DMSO (D), API-2 (A), roscovitine (R) or combination of API-2 and roscovitine (AR) for 8 hrs. Lysates were analysed by Western blotting. (FIG. 6B) Longer exposure to roscovitine reduces XIAP expression PC3 cells. Cells were exposed to drugs for 20 hrs. Lysates were analysed by Western blotting.

FIG. 7 illustrates the down-regulation of XIAP and inhibition of Akt activity induces apoptosis in PC3 cells. PC3 cells were incubated with control adenovirus or virus expressing siXIAP (Mohapatra et al. 2005) for 24 hrs. Cells received API-2 for 16 hrs. Lysates were analysed by Western blotting. For comparison, PC3 cells treated with roscovitine and API-2 is shown.

FIG. 8 illustrates the depletion of Cdk9 induces apoptosis of API-2 treated PC3 cells. PC3 cells were transfected twice with ON Targeted plus siRNAs (Dharmacon) corresponding to Cdk1, Cdk2, Cdk7 and Cdk9 or combination thereof using manufacturer's instruction. 48 hrs after the second transfection, cells were replated and treated with 20 mM API-2 for 12 hrs. Apoptosis was determined by the analysis of DNA fragmentation using Cell Death ELISA (Roche). Amounts of CDKs and b-actin were determined by Westerbn blotting.

### DETAILED DESCRIPTION OF THE PREFERRED EMBODIMENT

The present invention provides for methods of treating cancer in an individual in need thereof by administering to the individual an effective amount of an inhibitor of the P3K/Akt pathway in combination with roscovitine. Non-limiting examples of inhibitors that can be used to inhibit the P3K/Akt pathway include API-2 (Triciribine),

We show that rosc/LY and rosc/AP induce the apoptosis of androgen-dependent (LNCaP) and androgen-independent (PC3) prostate cancer cells. Two critical discoveries have been made by the inventors. First, cells that respond to roscovitine alone (LNCaP) initiate apoptosis sooner when co-treated. Second, cells that do not respond to roscovitine alone (PC3) apoptose when co-treated, although with delayed kinetics. In the absence of roscovitine, AKT inhibitors had no effect on LNCaP or PC3 survival, and in both cell lines, the combined treatment activated the mitochondrial pathway of apoptosis. Importantly, normal epithelial cells (RPWE) remained viable in the presence of roscovitine and AKT inhibitors. We identify events elicited by roscovitine (down-regulation of XIAP) and AKT inhibitors (accumulation of Bim) in LNCaP and PC3 cells. Additional data show that PC3 cells apoptose when treated with AKT inhibitors and depleted of either XIAP or Cdk9.

The programmed cell death or apoptosis requires activation of initiator caspases (members of cysteine aspartyl proteases), which in turn activates effector caspases that leads to cleavages and activation of executioner caspases, such as caspase-3, which ultimately results in cell death (e.g., plasma membrane blebbing and DNA fragmentation) [Strasser, A., L. O'Connor, and V. M. Dixit, 2000; Chang, H. Y. and X. Yang, 2000; Borner, C., 2003]. Apoptosis can be initiated via the interaction of death receptors (extrinsic pathway) with the adaptor proteins such as FADD and TRADD that activate initiator caspase-8, or through the mitochondrial pathway (intrinsic pathway) that requires disruption of the mitochondrial membrane and release of mitochondrial proteins, including Smac/DIABLO, Omi/HtrA2 and cyctochrome c. The latter associates with the adaptor protein Apaf-1 and activates initiator caspase-9. Most drugs induce apoptosis through the mitochondrial pathway [Strasser, A., L. O'Connor, and V. M. Dixit, 2000; Chang, H. Y. and X. Yang, 2000; Bomer, C., 2003; Hakem, R., et al., 1998; Kuida, K., et al., 1998]. Data presented here show that the combination of roscovitine and AKT inhibitors activates a mitochondrial pathway of apoptosis. Proteins that modulate caspase activity include the Bcl-2 proteins, IAPs (Inhibitor of Apoptosis Proteins), and p53.

Bcl-2 family: Members of the Bcl-2 family of proteins are critical determinants of mitochondria-dependent caspase activation [Bomer, C., 2003; Burlacu, A., Regulation of apoptosis by Bcl-2 family proteins. J Cell Mol Med, 2003]. The pro-survival members of the Bcl-2 family include Bcl-2, BC1-XL, Bcl-w and Mc1₁. The pro-apoptotic members of the Bcl-2 family comprise the multidomain apoptotic group (Bax, Bak and Bok) and the BH3-only proteins (Bad, Bid, Bim, Bmf, Noxa and Puma) [Cheng, E. H., et al., 2001; Zong, W. X., et al., 2001]. In healthy cells, Bax exists in an inactive form in the cytosol, whereas Bak resides in the mitochondria. When activated (i.e., homo-oligomerized), Bax and Bak perforate mitochondrial membranes to release cytochrome c, Smac/DIABLO, and Omi/HtrA2 and trigger the activation of caspases [Wei, M. C., et al., 2001]. BH3-only proteins, Bim and Bid, interact with and promote the oligomerization of Bax and Bak; others (e.g., Bad) displace Bim, Bid and Bak from anti-apoptotic Bcl-2 proteins (e.g., Bcl-2, BC1-XL, MCd-1) [Cheng, E. H., et al., 2001; Kuwana, T., et al., 2005; Yamaguchi, H. and H. G. Wang, 2002; Yang, E., et al., 1995]. Anti-apoptotic Bcl-2 proteins (which are mitochondrial) sequester Bim and Bid and sequester Bak to prevent its association with Bim and Bid.

IAP family (XIAP): The IAP family includes cIAP-1, cIAP-2, XIAP, and survivin [Vaux, D. L. and J. Silke, 2003; Holcik, M., H. Gibson, and R. G. Korneluk, 2001]. Of these proteins, XIAP is the most potent. The IAPs interact with and inhibit the activity of processed caspases; thus, they function as ‘brakes’ that can inhibit the apoptotic process once it begins. IAPs interact with both initiator (caspase-9 but not caspase-8) and effector (caspase-3) caspases [Deveraux, Q. L., et al., 1997; Srinivasula, S. M., et al., 2001]. Studies in our laboratory have shown that roscovitine reduces XIAP expression in LNCaP, LNCaP-Rf, and PC3 cells [Mohapatra, S., et al., 2005]. Over-expression of XIAP blocks the apoptosis of roscovitine-treated LNCaP cells and of glioma cells co-treated with roscovitine and the death receptor ligand TRAIL [Mohapatra, S., et al., 2005, Kim, E. H., et al., 2004]. Depletion of XIAP does not induce LNCaP and PC3 apoptosis (Preliminary Data) [Mohapatra, S., et al., 2005]; however, the XIAP inhibitor Smac and cytochrome c cooperatively activate caspases when co-injected into LNCaP cells [Carson, J. P., et al., 2002]. These findings suggest that loss of XIAP is necessary but insufficient for apoptosis. Of interest are studies showing enhanced resistance of metastatic prostate cancer cells to anoikis by ectopic expression of XIAP [Berezovskaya, O., et al., 2005].

P53: p53 accumulates in cells in response to many chemotoxic drugs, most strikingly in the nucleus [Vogelstein, B., D. Lane, and A. J. Levine, 2000]. Accumulation typically results from reductions in the abundance of Mdm2, which ubiquinates p53 in the nucleus and targets p53 for destruction [Haupt, Y., 2004, Haupt, Y., et al., 1997]. p53 promotes apoptosis by two mechanisms: it transactivates genes that encode apoptotic proteins such as Bax, and it translocates to mitochondria where it promotes cytochrome c release by interacting with Bcl-2 family members and mitochondrial proteins [Chipuk, J. E., et al., 2004; Moll, U. M. and A. Zaika, 2001]. The events responsible for the mitochondrial accumulation of p53 remain to be determined as do other aspects of p53-dependent, transcription-independent apoptosis. p53 mutations are fairly common in advanced prostate tumors and correlate with poor prognosis [Dong, J. T., 2006; Thomas, D. J., et al., 1993].

Studies in our laboratory have shown that roscovitine increases p53 abundance in LNCaP and LNCaP-Rf cells [Mohapatra, S., et al., 2005]. In support of p53 involvement in roscovitine-induced apoptosis, we have shown that: (i) prostate cancer cells expressing mutant p53 (DU145) are relatively resistant to roscovitine as are PC3 cells; (ii) DU145 and PC3 cells efficiently apoptose when supplied with roscovitine and wild-type p53; (iii) melanoma cells expressing wild-type p53 (A375, 888-Mel, 624-Mel) apoptose in response to roscovitine whereas those expressing mutant p53 do not (SK-Mel-2, SK-Mel-28, MeWo); and (iv) pifithrin-α rescues LNCaP cells from roscovitine-induced apoptosis [Mohapatra, S., et al., 2005; Mohapatra, S., et al., 2007]. Pifithrin-α inhibits p53 accumulation, transcription, and mitochondrial translocation [Dagher, P. C., 2004, Lorenzo, E., et al., 2002]. Data presented herein implicate transcription-independent actions of p53 in the apoptosis of roscovitine-treated LNCaP cells.

Roscovitine and Apoptosis: Cell cycle dysregulation is one of the hallmarks of malignant transformation, and thus, CDKs have emerged as attractive targets for cancer therapy [Senderowicz, A. M., 2003; Shapiro, G. I., 2006]. One class of small molecular CDK modulators includes roscovitine [2-(1-ethyl-2-hydroxyethylamino)-6-benzylamino-9-isopropylpurine] (also known as CYC202), which is a potent inhibitor of serine-threonine kinases: Cdk2, and Cdc2 that regulate cell cycle progression and Cdk7 and Cdk9 that regulate transcription [Meijer, L., et al., 1997; Wang, D., et al., 2001]. It inhibits CDK activity by directly competing for the ATP binding sites of CDKs and is highly selective; of the 151 kinases examined by Bach et al. [Bach, S., et al., 2005], R-roscovitine (the R-stereoisomer of roscovitine) bound only its known CDK targets and pyridoxal kinase, which phosphorylates vitamin B6. Roscovitine induces apoptosis of cells derived from a variety of tumor types [Mihara, M., et al., 2002; Wojciechowski, J., et al., 2003; Raje, N., et al., 2005; Tirado, O. M., S. Mateo-Lozano, and V. Notario, 2005] Mohapatra, 2007 in press; [Mohapatra, S., et al., 2005; Mohapatra, S., et al., 2007; Meijer, L., et al., 1997; Dai, Y., P. Dent, and S. Grant, 2002; McClue, S. J., et al., 2002; Senderowicz, A. M., 2003]. We have shown that roscovitine readily induces apoptosis in prostate cancer cells (LNCaP, LNCaP-Rf, but not PC3 and DU145 cells), melanoma cells (A375, 888, 624) and HTLV-I transformed T cell lines (MT-2). It is not clear, how roscovitine induces apoptosis. However, our preliminary results implicate Cdk9 as one of the CDKs that may be a direct target of roscovitine in PC3 cells. Moreover, results of our study and others implicate that roscovitine targets two other proteins: XIAP and p53 in many tumor cells [Mohapatra, S., et al., 2005; Kim, E. H., et al., 2004; Wojciechowski, J., et al., 2003; David-Pfeuty, T., 1999; Hahntow, I. N., et al., 2004; Kotala, V., et al., 2001; Lu, W., et al., 2001; Mohapatra, S., et al., 2003] (Mohapatra, 2007 in press).

Roscovitine clears slowly from plasma via oxidative metabolism of the side chain hydroxyl group to form carboxylic acid and is subsequently excreted in urine [Raynaud, F. I., et al., 2004; Raynaud, F. I., et al., 2005; Nutley, B. P., et al., 2005]. It has better tissue distribution, and has the highest tumor uptake [Raynaud, F. I., et al., 2004; Raynaud, F. I., et al., 2005; Nutley, B. P., et al., 2005]. Roscovitine can be given orally, 3 times a day at 200 mg/kg or 2 times a day at 500 mg/kg, to sustain therapeutic exposure without any toxicity or adverse effects. A more recent Phase I trial of patients with malignant tumors refractory to conventional treatments showed toxicity at higher doses of R-roscovitine and no objective tumor responses [Benson, C., et al., 2007]. Phase II clinical trials for R-roscovitine for lung, breast, and B cell cancer showed limited toxicity and partial responses lasting more than four months [Fischer, P. M. and A. Gianella-Borradori, 2005].

Regulation of PI3K/AKT signaling in prostate cancer: AKT (aka protein kinase B) belongs to a family of PI3K-regulated serine/threonine (Ser/Thr) kinases [Franke, T. F., et al., 1995; Burgering, B. M. and P. J. Coffer, 1995; Cross, D. A., et al., 1995]. Several lines of evidence suggest that the PI3K/AKT pathway plays a central role in the development and progression of prostate cancer and other malignancies [Burgering, B. M. and P. J. Coffer, 1995; Majumder, P. K. and W. R. Sellers, 2005; Li, L., et al., 2005]. Inactivation of PTEN (phosphatase and tensin homolog deleted from chromosome 10), either by loss of heterozygousity or mutational silencing, was found in many human cancers, including glioblastomas, endometrial cancers and 63% of advanced prostate cancers [Burgering, B. M. and P. J. Coffer, 1995; Majumder, P. K. and W. R. Sellers, 2005; Li, L., et al., 2005]. This results in accumulation of the phosphoinositol-(3, 4, 5)-triphosphate (PIP3), which in turn binds to the pleckstrin homology (pH) domain of Ser/Thr kinase AKT, leading to the recruitment of AKT to the cell membrane [Burgering, B. M. and P. J. Coffer, 1995]. A conformational change of AKT results in phosphorylation of residues Thr-308 and Ser-473 by upstream kinases, PDK-1 and PDK-2 or integrin linked kinase, respectively [Vanhaesebroeck, B. and D. R. Alessi, 2000; Stocker, H., et al., 2002]. AKT has three isoforms, AKT1, AKT2 and AKT3, which are closely related. AKT1 and AKT2 seems to be expressed ubiquitously, whereas AKT3 expression seems to be more restricted [Chan, T. O., S. E. Rittenhouse, and P. N. Tsichlis, 1999]. Full activation of the AKT requires phosphorylation at Thr³⁰⁸ (AKT1), Thr³⁰⁹ (AKT2) or Thr³⁰⁵ (AKT3) in the activation loop and Ser⁴⁷³ (AKT1), Ser⁴⁷⁴ (AKT2) or Ser⁴⁷² (AKT3) in the C-terminal activation domain [Datta, S. R., A. Brunet, and M. E. Greenberg, 1999]. AKT regulates cell growth, cell cycle progression, cell survival, migration, epithelial-mesenchymal transition and angiogenesis by inactivating its down-stream substrates [Datta, S. R., A. Brunet, and M. E. Greenberg, 1999; Martelli, A. M., et al., 2006].

The PI3K/AKT signaling pathway is a predominant growth factor survival pathway in prostate cancer cells. Specifically, prostate cancer cells lacking active PTEN or PTEN-null cells remain dependent upon activation of the PI3K pathway for growth and survival. Reconstitution of active PTEN to such cells either arrests cells in G1 or induces apoptosis [Yuan, X. J. and Y. E. Whang, 2002]. Phosphorylated AKT (p-Akt) is seen in prostate cancer specimens with a high Gleason score [Liao, Y., et al., 2003]. Moreover, prostate cancer cell lines that have been obtained from metastatic lesions (e.g., LNCaP, PC3) or that are strictly androgen-independent (e.g., 22RV-1, C4-2) harbor point mutations or deletions of PTEN and express a higher basal level of p-Akt than do PTEN wild-type (wt) cells [van Bokhoven, A., et al., 2003]. Further, it has also been shown that in LNCaP cells, androgen ablation alone increases PI3K/AKT activation. The increased PI3K/AKT signaling was necessary for survival of acute and chronic androgen deprivation [Murillo, H., et al., 2001]. Continued dependence of advanced prostate cancer cells on the PI3K/AKT signaling pathway provides a notable therapeutic opportunity.

AKT Inhibitors and Apoptosis.

Pharmacological inhibitors of PI3K, such as LY294002 and Wortmanin, which target the p110 catalytic subunit of PI3K, induce a potent apoptotic response in most prostate cancer cell lines, including LNCaP, LAPC4 and LAPC9 [Lin, J., et al., 1999]; however, these molecules have relatively broad specificity and short in vivo half-life and are poorly suited for clinical development [Majumder, P. K. and W. R. Sellers, 2005]. Further, the dosages required to induce apoptosis of prostate cancer cells are considerably high and difficult to achieve for human trials [Lin, J., et al., 1999]. Peptidomimetics have been successful in blocking the recruitment of the PI3K to receptor tyrosine kinases by disrupting the phosphotyrosine binding of the SH2-domain of the p85 subunit of PI3K. However, these agents have not yet been tested in vivo [Eaton, S. R., et al., 1998]. Inhibitors of AKT have been attractive for years. Through combinatorial chemistry, high-throughput and virtual screening and traditional medicinal chemistry, a number of inhibitors of the AKT pathway have been identified [Barnett, S. F., M. T. Bilodeau, and C. W. Lindsley, 2005; DeFeo-Jones, D., et al., 2005]. We have been working with one of the AKT inhibitors, API-2, which was discovered by screening the NCI Diversity set comprising of 140,000 compounds [Yang, L., et al., 2004; Cheng, J. Q., et al., 2005]. API-2 suppressed the kinase activity and phosphorylation level of all three AKT family members by inhibiting the interaction with the pH domain. API-2 is highly selective for AKT and does not inhibit PI3K, PDK1, PKC, SGK, PKA, Stat3, Erk-1/2 or JNK [Yang, L., et al., 2004]. Previous studies have shown that API-2 (Triciribine) inhibits DNA synthesis and has anti-tumor and anti-viral activity [Wotring, L. L., et al., 1990]. However in clinical trials, API-2 (Triciribine), when administered at very high dosages, had significant side effects, including hepatotoxicity, hypertriglyceridemia, thrombocytopenia, and hyperglycemia that hampered its clinical use [Feun, L. G., et al., 1984; Feun, L. G., et al., 1993]. In contrast, a recent study suggests that API-2, when administered at a dosage of 1 mg/kg/day, had no detectable toxicity and it potently inhibited tumor growth in nude mice of ovarian cancer xenografts in which AKT is aberrantly expressed/activated [Yang, L., et al., 2004; Cheng, J. Q., et al., 2005]. No detectable side effects were observed in these mice. Together, these results indicate that API-2 (Triciribine), at a low dose, could achieve anti-tumor growth without significant side effects.

AKT inhibitors themselves are not apoptotic for LNCaP cells [Yuan, X. J. and Y. E. Whang, 2002; Carson, J. P., G. Kulik, and M. J. Weber, 1999]. They do, however, promote LNCaP apoptosis in conjunction with serum deprivation, death receptor ligands, and DNA-damaging drugs [Yuan, X. J. and Y. E. Whang, 2002]. In contrast to LNCaP cells, androgen-independent cells (PC3 and LNCaP-abl) do not readily succumb to AKT inhibitors, even in serum-free medium. Our results, therefore, are noteworthy: we show efficient apoptosis of PC3 cells co-treated with roscovitine and AKT inhibitors in the presence of serum and accelerated apoptosis of co-treated as compared with roscovitine-treated LNCaP cells. How AKT inhibitors facilitate the mitochondria-dependent apoptosis of prostate cancer cells remains to be determined. Potential mechanisms include accumulation of the BH3-only protein Bim and dephosphorylation and activation of Bad. The FOXO factors activate the Bim promoter, and AKT phosphorylates Bim, which may prevent its degradation. In the study of Sastry et al., knockdown of Bad blocked the apoptosis of serum-starved, LY294002-treated LNCaP cells. As described below, LY294002 and API-2 increase amounts of Bim in LNCaP and PC3 cells.

Definitions

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay occurrence and/or recurrence. An effective amount can be administered in one or more doses. In the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

As used herein, “treatment” refers to obtaining beneficial or desired clinical results. Beneficial or desired clinical results include, but are not limited to, any one or more of: alleviation of one or more symptoms (such as tumor growth or metastasis), diminishment of extent of cancer, stabilized (i.e., not worsening) state of cancer, preventing or delaying spread (e.g., metastasis) of the cancer, preventing or delaying occurrence or recurrence of cancer, delay or slowing of cancer progression, amelioration of the cancer state, and remission (whether partial or total). The methods of the invention contemplate any one or more of these aspects of treatment.

A “subject in need of treatment” is a mammal with cancer that is life-threatening or that impairs health or shortens the lifespan of the mammal.

A “pharmaceutically acceptable” component is one that is suitable for use with humans and/or animals without undue adverse side effects (such as toxicity, irritation, and allergic response) commensurate with a reasonable benefit/risk ratio.

A “safe and effective amount” refers to the quantity of a component that is sufficient to yield a desired therapeutic response without undue adverse side effects (such as toxicity, irritation, or allergic response) commensurate with a reasonable benefit/risk ratio when used in the manner of this invention.

A “pharmaceutically acceptable carrier” is a carrier, such as a solvent, suspending agent or vehicle, for delivering the compound or compounds in question to the animal or human. The carrier may be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated.

Compositions

Roscovitine and R-roscovitine (CYC002) are potent inhibitors of the activity of cyclin-dependent kinases (CDKs), most notably cdk2, cdk1, and cdk7, as well as cdk5 and cdc2 [Meijer L, et al. Eur J Biochem 1997;243:527-536; McClue S J, et al., Int J Cancer 2002; 102:463-468]. Roscovitine is the compound 6-benzylamino-2-[(R)-1-ethyl-2-hydroxye-thylamino]-9-isopropylpurine. R-roscovitine refers to the R enantomer of roscovitine, specifically the compound 2-(1-R-hydroxymethylpropylamino)-6-benzylamino-9-iso-propylpurine. As used in the claims, “roscovitine” refers to both the (R) and (S) stereoisomers, as well as salts and prodrugs thereof.

Roscovitine is typically administered from about 0.05 to about 5 g/day, preferably from about 0.4 to about 3 g/day. Roscovitine is preferably administered orally in tablets or capsules. The total daily dose of roscovitine can be administered as a single dose or divided into separate dosages administered two, three or four time a day.

API-2 or Triciribine (TCN) (see also triciribine 5′-phosphate (TCN-P), and the DMF adduct of triciribine (TCN-DMF)) is a known compound having the formula:

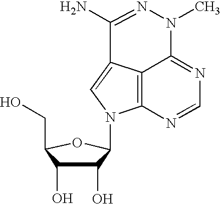

As used in the claims, API-2 refers generally to TCN, TCN-P, TCN-DMF, and pharmaceutically acceptable salts and prodrugs thereof. TCN may be synthesized as described in Tetrahedron Letters, vol. 49, pp. 4757-4760 (1971). TCN-P may be prepared as described in U.S. Pat. No. 4,123,524. TCN-DMF is described in INSERM, vol. 81, pp. 37-82 (1978).

Although the exact dosage of TCN, TCN-P, TCN-DMF, or a pharmaceutically acceptable salt thereof to be administered will vary according to the size and condition of the patient, a suitable dosage range is 15 to 350 mg/m² of body surface, preferably 15 to 96 mg/m² of body surface, most preferably 25 to 50 mg/m² of body surface.

The TCN, TCN-P, TCN-DMF, or pharmaceutically acceptable salt thereof may be administered according to the present invention by any suitable route, such as intravenously, parenterally, subcutaneously, intramuscularly, or orally. The TCN, TCN-P, TCN-DMF, or pharmaceutically acceptable salt thereof may be administered in any conventional form such as a pharmaceutical composition. Suitable pharmaceutical compositions are those containing, in addition to TCN, TCN-P, TCN-DMF, or pharmaceutically acceptable salt thereof, a pharmaceutically acceptable carrier, such as water, starch, sugar, etc. The composition may also contain flavoring agents and may take the form of a solution, tablet, pill, capsule, etc. The ratio of the weight of TCN, TCN-P, TCN-DMF, or pharmaceutically acceptable salt thereof to the weight of the pharmaceutical composition may, of course, vary but is suitably within 1:1 to 1:5000.

For purposes of the present invention, the term pharmaceutically acceptable salt thereof refers to any salt of TCN, TCN-P, or TCN-DMF which is pharmaceutically acceptable and does not greatly reduce or inhibit the activity of TCN, TCN-P, or TCN-DMF. Suitable examples for TCN and TCN-DMF include acid addition salts, with an organic or inorganic acid such as acetate, tartrate, trifluoroacetate, lactate, maleate, fumarate, citrate, methane sulfonate, sulfate, phosphate, nitrate, or chloride. Suitable examples of salts for TCN-P include those in which one or more of the acidic phosphate hydrogens has been replaced with an ion, such as sodium, potassium, calcium, iron, ammonium, or mono-, di- or tri-lower-alkyl ammonium, in addition to the acid addition salts described above. It is to be further understood that the terms TCN, TCN-P, TCN-DMF, and pharmaceutically acceptable salts thereof include all the hydrated forms of these compounds as well as the anhydrous forms.

Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Combinations

In one preferred embodiment of the invention, one or more Akt inhibitors (e.g. API-2) is administered in combination with one or more XIAP inhibitors (e.g. roscovitine). In such cases, the compounds of the invention may be administered consecutively, simultaneously or sequentially with the one or more other PI3K inhibitors.

It is known in the art that many drugs are more effective when used in combination. In particular, combination therapy is desirable in order to avoid an overlap of major toxicities, mechanism of action and resistance mechanism(s). Furthermore, it is also desirable to administer most drugs at their maximum tolerated doses with minimum time intervals between such doses. The major advantages of combining drugs are that it may promote additive or possible synergistic effects through biochemical interactions and also may decrease the emergence of drug resistance which would have been otherwise responsive to initial treatment with a single agent.

Beneficial combinations may be suggested by studying the activity of the test compounds with agents known or suspected of being valuable in the treatment of a particular disorder. This procedure can also be used to determine the order of administration of the agents, i.e. before, simultaneously, or after delivery.

It is to be understood that the present method includes embodiments in which TCN, TCN-P, TCN-DMF, or pharmaceutically acceptable salt thereof is administered to a patient who is also receiving roscovitine. The present compound(s) and roscovitine may be administered to the patient in a single composition comprising both the present compounds and roscovitine. Alternatively, the present compound(s) and roscovitine may be administered separately. Further, the present method includes embodiments in which roscovitine is administered, without TCN, TCN-P, TCN-DMF, or a pharmaceutically acceptable salt thereof, for a suitable time period of hours, days, or weeks, and the roscovitine therapy is either preceded or followed by administration of TCN, TCN-P, TCN-DMF, or a pharmaceutically acceptable salt, either with or without roscovitine.

As recited above the method and treatment combination of the present invention also includes at least one of an Akt inhibitor. Generally any Akt inhibitor, that is, any pharmaceutical agent having specific Akt inhibitor activity may be utilized in the present invention. Such Akt inhibitors are described, for instance, in US20060104951A1 to Mountz et al., WO05046678A1 TO DEV ET AL. Additional Akt inhibitors are described in WO2002083064, WO2002083138, WO2002083140, WO2002083139, WO2002083675, WO2003010281, WO200198290, WO03014090, WO200248114, WO2003013517, WO200230423, WO2002057259, WO200222610, WO2003011854, WO2003084473, and WO2003011855, which patent applications are herein incorporated by reference to the extent of their disclosure of Akt inhibitor compounds and methods of making and using the same.

The present invention provides for an Akt inhibitor; wherein the Akt inhibitor is a molecule illustratively including a cyclooxygenase-2 inhibitor, a pyridinyl imidazole inhibitor, a Ber-Abl tyrosine kinase inhibitor and a PI-3 kinase inhibitor. An example of a pyridinyl imidazole is SB203580 commercially available from Calbiochem-Novabiochem. An example of a Ber-Abl tyrosine kinase inhibitor is CGP57148B, also known as STI-571, made by Novartis Pharma AG. An example of a PI-3 kinase inhibitor is LY294002, also known as 2-(4-morpholinyl)-8-phenyl-4H-1-benzopyran-4-one, commercially available from Calbiochem. An example of a cyclooxygenase-2 inhibitor is celecoxib.

It will be appreciated by those skilled in the art that Akt inhibition is achieved by inhibition of factors that cause an increase in Akt levels, activity or phosphorylation or which are necessary for Akt activation. Factors known to increase Akt or which are necessary for Akt activation illustratively include insulin-like growth factor-1, IL-1, PDGF, focal adhesion kinase, lipoarabinomannan and Syk.

Pharmaceutical Compositions

Although roscovitine and/or API-2 (or a pharmaceutically acceptable salt, ester or pharmaceutically acceptable solvate thereof) can be administered alone, for human therapy it will generally be administered in admixture with a pharmaceutical carrier, excipient or diluent.

An embodiment of the invention therefore relates to the administration in combination with a pharmaceutically acceptable excipient, diluent or carrier.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985). Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Salts/Esters

The active agent of the present invention can be present in the form of a salt or an ester, in particular a pharmaceutically acceptable salt or ester.

Pharmaceutically acceptable salts of the active agent of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, e.g. by a halogen).

Enantiomers/Tautomers

The invention also includes where appropriate all enantiomers and tautomers of the active agent. The man skilled in the art will recognize compounds that possess optical properties (one or more chiral carbon atoms) or tautomeric characteristics. The corresponding enantiomers and/or tautomers may be isolated/prepared by methods known in the art.

Stereo and Geometric Isomers

The active agent of the invention may exist in the form of different stereoisomers and/or geometric isomers, e.g. it may possess one or more asymmetric and/or geometric centers and so may exist in two or more stereoisomeric and/or geometric forms. The present invention contemplates the use of all the individual stereoisomers and geometric isomers of the agent, and mixtures thereof. The terms used in the claims encompass these forms, provided said forms retain the appropriate functional activity (though not necessarily to the same degree).

The present invention also includes all suitable isotopic variations of the active agent or pharmaceutically acceptable salts thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agents of the present invention and pharmaceutically acceptable salts thereof can generally be prepared by conventional procedures using appropriate isotopic variations of suitable reagents.

Solvates

The present invention also includes solvate forms of the active agent of the present invention. The terms used in the claims encompass these forms.

Polymorphs

The invention furthermore relates to various crystalline forms, polymorphic forms and (an)hydrous forms of the active agent. It is well established within the pharmaceutical industry that chemical compounds may be isolated in any of such forms by slightly varying the method of purification and or isolation form the solvents used in the synthetic preparation of such compounds.

Prodrugs

The invention further includes the active agent of the present invention in prodrug form. Such prodrugs are generally compounds wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include esters (for example, any of those described above), wherein the reversion may be carried out be an esterase etc. Other such systems will be well known to those skilled in the art.

Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 2000 mg and more preferably from 50-1000 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredients can be incorporated into a cream consisting of an aqueous emulsion of polyethylene glycols or liquid paraffin. The active ingredients can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-500 mg, of active ingredient per dose.

Compositions may be formulated in unit dosage form, i.e., in the form of discrete portions containing a unit dose, or a multiple or sub-unit of a unit dose.

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods. See, generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.; as well as Guthrie et al., Guide to Yeast Genetics and Molecular Biology, Methods in Enzymology, Vol. 194, Academic Press, Inc., (1991), PCR Protocols: A Guide to Methods and Applications (Innis, et al. 1990. Academic Press, San Diego, Calif.), McPherson et al., PCR Volume 1, Oxford University Press, (1991), Culture of Animal Cells: A Manual of Basic Technique, 2nd Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.).

The invention is described below in examples which are intended to further describe the invention without limitation to its scope.

Prostate cancer cell lines used in our studies include LNCaP, LNCaP-Rf, PC3 and PC3-MM2. LNCaP cells are androgen-dependent, and LNCaP-Rf cells are androgen-independent derivatives of LNCaP cells; both cell lines express wild-type p53 [Horoszewicz, J. S., et al., 1980; Zegarra-Moro, O. L., et al., 2002]. PC3 cells are androgen-independent and ‘p53-null’ (i.e., they express a truncated, unstable form of p53 not detected in cell lystates) [Kaighn, M. E., et al., 1979]. PC3-MM2 cells are a metastatic variant of PC3 cells [Pettaway, C. A., et al., 1996]. PC3-T55 cells are resistant to taxol [Patterson, S. G., et al., 2006]. LNCaP cells and PC3 cells were derived from a lymph node metastasis and a bone metastasis, respectively, of human prostate tumors. All these cell lines express constitutively active AKT. To distinguish the effects of our therapeutics between normal and tumor cells, we also used an immortalized but non-tumorigenic prostate epithelial cell line, RWPE, in our study. All experiments were performed on growing cells in medium containing 10% fetal calf serum.

### EXAMPLE 1

### Roscovitine and AKT Inhibitors Induce Rapid Onset of Apoptosis of LNCaPs

We previously reported that roscovitine can induce apoptosis of LNCaP and LNCaP-Rf prostate cancer cells, but not PC3 cells that are p53 null [Mohapatra, S., et al., 2005]. Apoptosis required accumulation of p53 and down-regulation of XIAP.

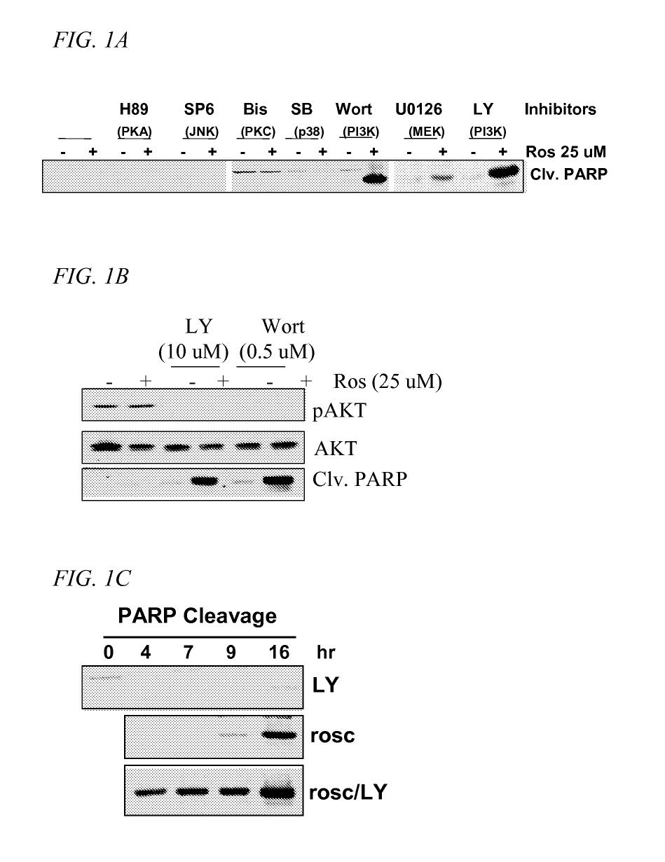

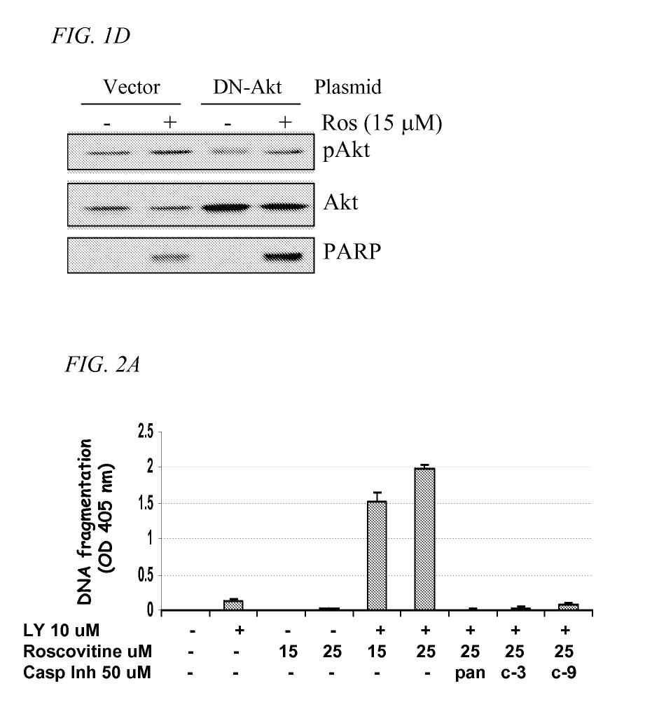

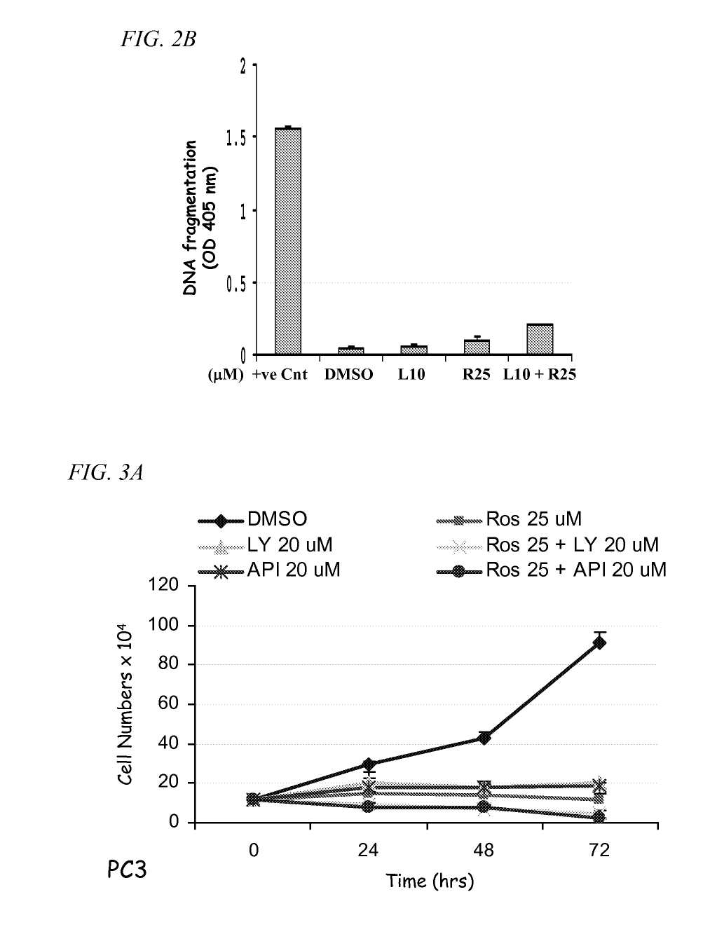

In an effort to determine whether inhibitors of key signaling pathways, such as the protein kinase A, Janus kinase, protein kinase C, p38, PI3K or Map kinase pathway, can enhance roscovitine-induced apoptosis, we treated LNCaP cells with a combination of roscovitine and one of the pathway specific inhibitors for 8 hours and examined the cell lysates for apoptosis. Apoptosis was monitored by cleavage of the caspase-3 substrate poly (ADP-ribose) polymerase (PARP). Results show that the combination of roscovitine with wortmannin or LY, inhibitors of PI3K, induced significant PARP cleavage (FIGS. 1A-B). By themselves, roscovitine, wortmannin or LY did not induce any PARP cleavage. Robust PARP cleavage was evident in cells exposed to roscovitine for more than 9 hr and in cells exposed to rosc/LY for as little as 4 hr (FIG. 1C). At 16 hr, amounts of cleaved PARP were similar in co-treated and roscovitine-treated cells. Thus, co-treated cells apoptose much sooner than do cells receiving roscovitine alone; however, the magnitude of the response is similar in both populations. Additional data show enhanced PARP cleavage in LNCaP cells exposed to lower dose of roscovitine (15 uM) for 16 hr in the presence as compared with the absence of dominant-negative AKT (FIG. 1D). LNCaP cells did not apoptose when exposed to rosc/LY in the presence of a caspase-9 or caspase-3 inhibitor (FIG. 2a). These results suggest that the combination treatment activates the mitochondrial pathway of apoptosis in LNCaPs. In contrast to LNCaPs, normal prostate epithelial cell (RWPE) remained viable in the presence of the inhibitors, both alone and in combination (FIG. 2A).

### EXAMPLE 2

### Roscovitine and AKT Inhibitors Cooperate to Induce PC3 Apoptosis

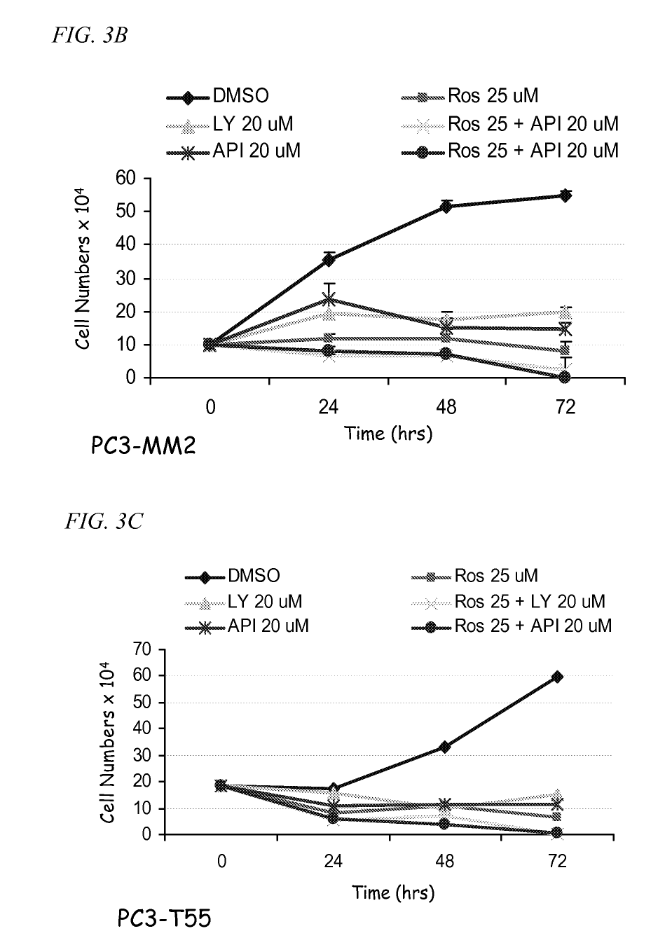

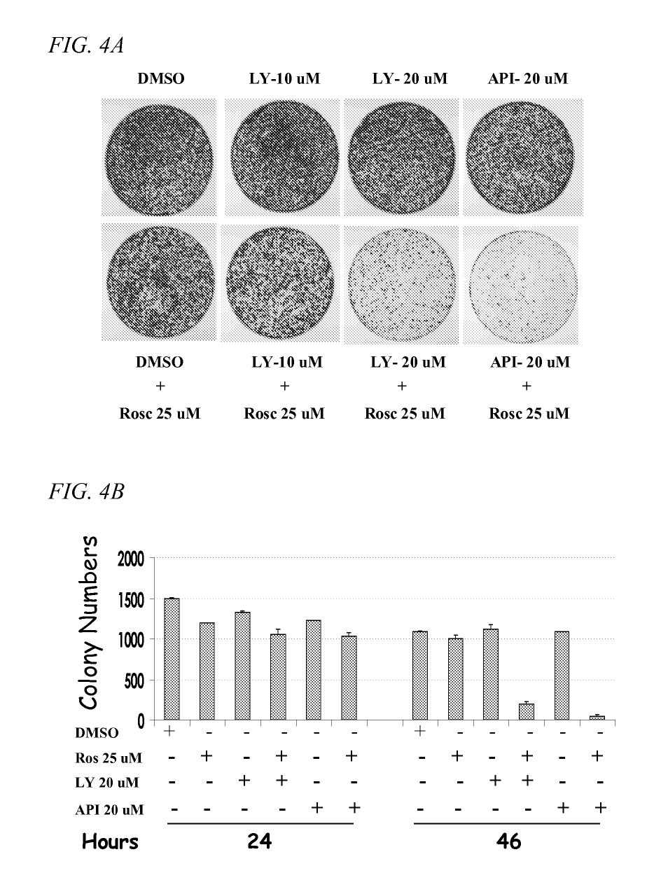

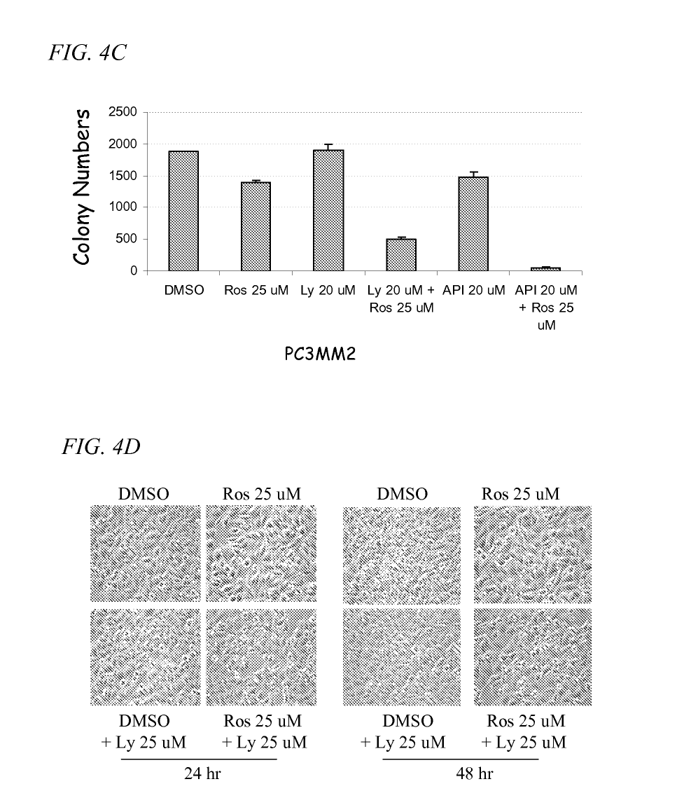

PC3 and PC3-MM2 cells received roscovitine, LY, API-2, rosc/LY or rosc/AP for 72 hr and alterations in cell growth were monitored. As shown in FIG. 3 A-B, LY, API, roscovitine or their combinations inhibit cell growth. However, inhibition of cell growth below the initial plating density was observed when cells were exposed to rosc/LY or rosc/API. Similar growth inhibition was also observed in PC3-T55 cells that were resistant to taxol (FIG. 3C). Further, effects of the combination treatment in altering colony formation potential were examined. For this purpose, cells were exposed to drugs for 24 or 46 hrs and trypsinized. Ten thousand viable cells were replated and allowed to grow colonies for 2 weeks. Results shown in FIG. 4 A-C suggest that significant inhibition in colony formation was detected only when cells were exposed to rosc/LY or rosc/API for longer than 24 hrs. It is to be noted that, even after 48 hr exposure most of the cells were alive, as shown in FIG. 4D.

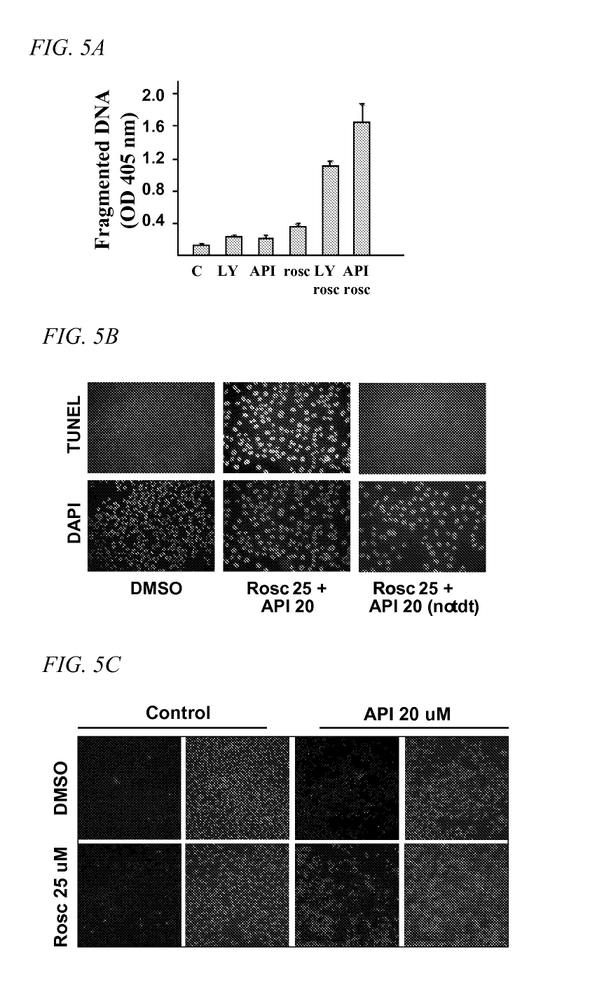

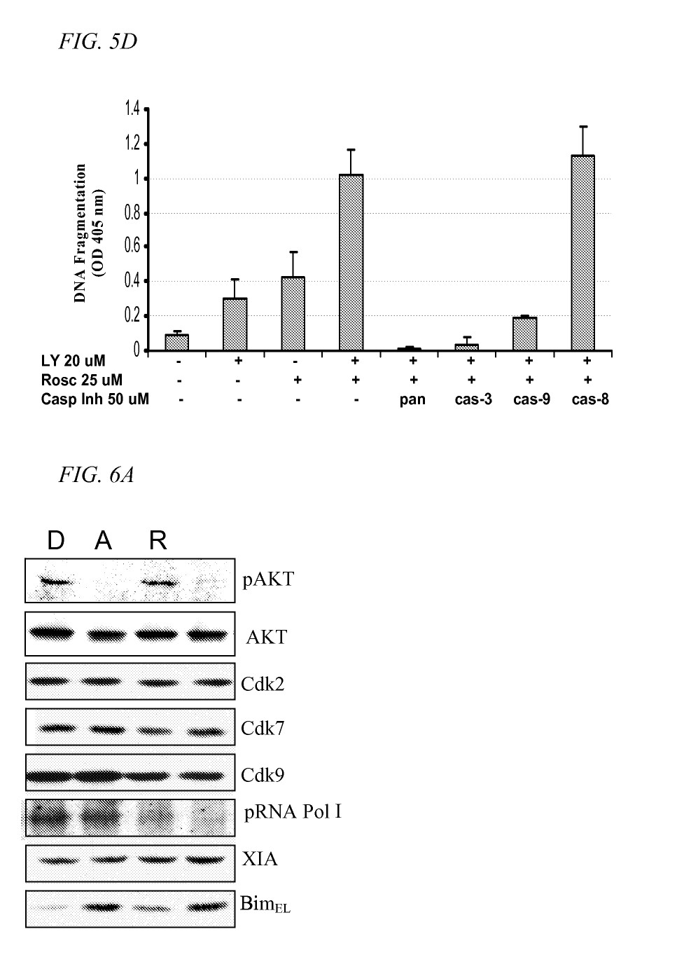

PC3 cells readily apoptosed when exposed to rosc/LY or rosc/AP but not when exposed to roscovitine, LY or API-2 alone (FIG. 5A-B). The response obtained with the combination treatment was similar in magnitude in LNCaP and PC3 cells, although PC3 cells required a longer exposure time. A greater than 70% of cells co-treated with rosc/AP were positive for activated caspases as determined by FLICA assay as compared with less than 15% in the other conditions (FIG. 5C). Moreover, PC3 cells did not apoptose when exposed to rosc/LY in the presence of a caspase-9 or caspase-3 inhibitor (FIG. 5D); Caspase-8 inhibitor did not have any effect. Collectively, the data in FIG. 5 show that PC3 apoptosis requires both CDK inactivation and abrogation of AKT signaling. A similar result was obtained in PC3-MM2 and PC3-T55 cells (data not shown).

### EXAMPLE 3

### Downstream Mediators of Apoptosis

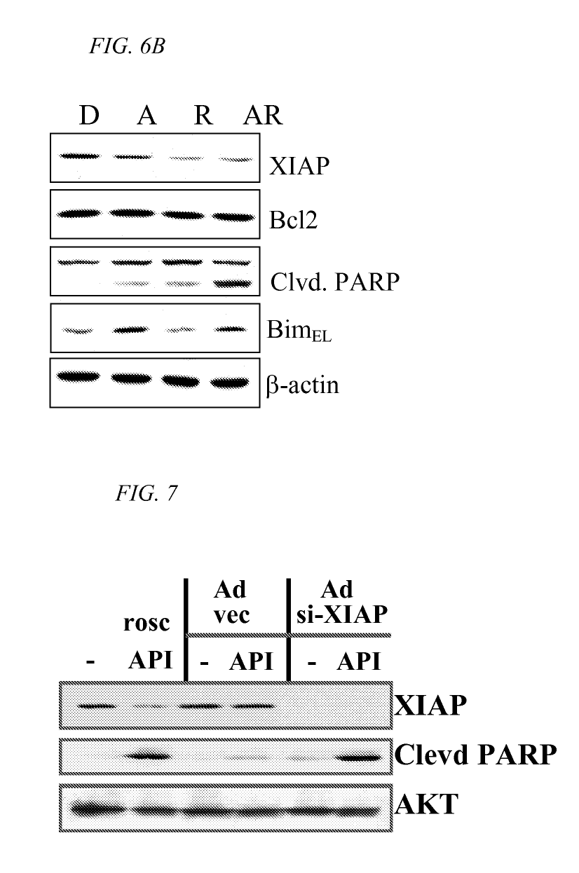

XIAP and Bim. As anticipated, short-term exposure to roscovitine did not alter expression of Cdk2, Cdk7 or Cdk9. However, it reduced activity of Cdk7/Cdk9 as measured by phosphorylation of RNA-Pol II. API-2 reduced AKT activity and increased Bim abundance (FIG. 6A). Longer exposure to roscovitine reduced expression of XIAP, but not Bcl-2 (FIG. 6B) [Mohapatra, S., et al., 2005]. Roscovitine did not alter Bim abundance in either the presence or absence of API-2; API-2 slightly reduced XIAP abundance in the absence of roscovitine but had no effect in the presence of roscovitine. These findings identify XIAP as a potential roscovitine target and Bim as a potential API-2 target in PC3 cells. We note that there are several isoforms of Bim, most notably Bim_{S}, Bim_{L} and Bim_{EL}. In LNCaP and PC3 cells, Bim_{EL} was the predominant form.

To assess the relevance of XIAP down-regulation, we depleted PC3 cells of XIAP by RNA interference. Cells were infected with adenovirus alone or adenovirus encoding XIAP siRNA; infected cells received API-2 for 16 hr, and PARP cleavage was determined. XIAP siRNA (and consequent knockdown of XIAP) was not apoptotic per se (FIG. 7). However, XIAP siRNA in conjunction with API-2 elicited apoptosis as effectively as did rosc/AP. Thus, XIAP down-regulation is indeed consequential and perhaps represents the sole contribution of roscovitine to PC3 apoptosis.

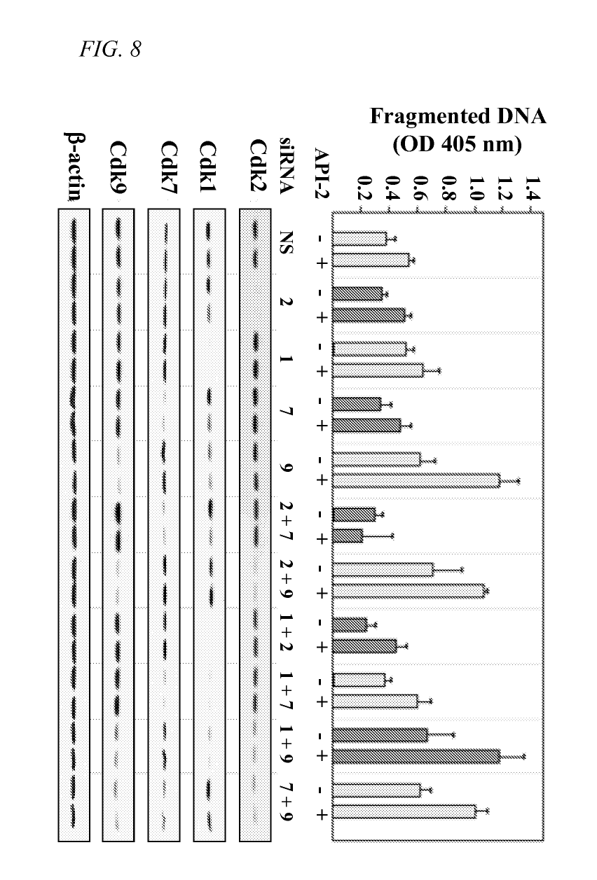

Cdk9. To identify the CDK whose inactivation signals the death of roscovitine-treated cells, we transfected PC3 cells with siRNA oligonucleotides to Cdk1, Cdk2, Cdk7 and Cdk9 either individually or in combination. Transfected cells received API-2 for 12 hr, and apoptosis was quantified by the DNA fragmentation assay. Western blots show specific depletion of the targeted CDK (FIG. 8). Knockdown of Cdk9 increased amounts of fragmented DNA in the presence (but not the absence) of API-2. Knockdown of Cdk2, Cdk1, and Cdk7 either alone or in combination did not affect the viability of untreated or API-2-treated cells. These findings identify Cdk9 as a proximal mediator of roscovitine in apoptotic signaling in PC3 cells. Cdk7 and Cdk9 promote distinct aspects of transcription (initiation and elongation, respectively); thus, it is unclear why depletion of Cdk7 did not affect survival. Cdk9 may phosphorylate the Cdk7 site (serine 5) of RNA polymerase II or residual amounts of Cdk7 in Cdk7-depleted cells may allow initiation of at least some transcripts. We note that the roscovitine does not globally inhibit transcription. Residual transcription may account for Bim accumulation in cells treated with rosc/LY or rosc/AP; an alternative mechanism involves continued translation of Bim mRNA and stabilization of Bim protein.

We show that rosc/LY and rosc/AP induce the apoptosis of androgen-dependent (LNCaP) and androgen-independent (PC3) prostate cancer cells. We propose two models: (1), cells that respond to roscovitine alone (LNCaP) initiate apoptosis sooner when co-treated; (2) cells that do not respond to roscovitine alone (PC3) apoptose when co-treated, although with delayed kinetics. In the absence of roscovitine, AKT inhibitors had no effect on LNCaP or PC3 survival, and in both cell lines, the combined treatment activated the mitochondrial pathway of apoptosis. Importantly, normal epithelial cells (RPWE) remained viable in the presence of roscovitine and AKT inhibitors. We identify events elicited by roscovitine (down-regulation of XIAP) and AKT inhibitors (accumulation of Bim) in LNCaP and PC3 cells. Additional data show: PC3 cells apoptose when treated with AKT inhibitors and depleted of either XIAP or Cdk9.

It has been shown that rosc/LY and rosc/AP induce the apoptosis of androgen-dependent (LNCaP) and androgen-independent (PC3) prostate cancer cells. Two critical discoveries have been made by the inventors based upon these showings. First, cells that respond to roscovitine alone (LNCaP) initiate apoptosis sooner when co-treated. Second, cells that do not respond to roscovitine alone (PC3) apoptose when co-treated, although with delayed kinetics. In the absence of roscovitine, AKT inhibitors had no effect on LNCaP or PC3 survival, and in both cell lines, the combined treatment activated the mitochondrial pathway of apoptosis. Importantly, normal epithelial cells (RPWE) remained viable in the presence of roscovitine and AKT inhibitors. Additionally, events elicited by roscovitine (down-regulation of XIAP) and AKT inhibitors (accumulation of Bim) in LNCaP and PC3 cells have been identified. Additional data show that PC3 cells apoptose when treated with AKT inhibitors and depleted of either XIAP or Cdk9.

### REFERENCES

Bach, S., et al., Roscovitine targets, protein kinases and pyridoxal kinase. J Biol Chem, 2005. 280 (35): p. 31208-19.
Barnett, S. F., M. T. Bilodeau, and C. W. Lindsley, The Akt/PKB family of protein kinases: a review of small molecule inhibitors and progress towards target validation. Curr Top Med Chem, 2005. 5(2): p. 109-25.
Benson, C., et al., A phase I trial of the selective oral cyclin-dependent kinase inhibitor seliciclib (CYC202; R-Roscovitine), administered twice daily for 7 days every 21 days. Br J Cancer, 2007. 96(1): p. 29-37.
Berezovskaya, O., et al., Increased expression of apoptosis inhibitor protein XIAP contributes to anoikis resistance of circulating human prostate cancer metastasis precursor cells. Cancer Res, 2005. 65(6): p. 2378-86.
Borner, C., The Bcl-2 protein family: sensors and checkpoints for life-or-death decisions. Mol Immunol, 2003. 39(11): p. 615-47.
Burgering, B. M. and P. J. Coffer, Protein kinase B (c-Akt) in phosphatidylinositol-3-OH kinase signal transduction. Nature, 1995. 376(6541): p. 599-602.
Burlacu, A., Regulation of apoptosis by Bcl-2 family proteins. J Cell Mol Med, 2003. 7(3): p. 249-57.
Carson, J. P., G. Kulik, and M. J. Weber, Antiapoptotic signaling in LNCaP prostate cancer cells: a survival signaling pathway independent of phosphatidylinositol 3′-kinase and Akt/protein kinase B. Cancer Res, 1999. 59(7): p. 1449-53.
Carson, J. P., et al., Smac is required for cytochrome c-induced apoptosis in prostate cancer LNCaP cells. Cancer Res, 2002. 62(1): p. 18-23.
Chan, T. O., S. E. Rittenhouse, and P. N. Tsichlis, AKT/PKB and other D3 phosphoinositide-regulated kinases: kinase activation by phosphoinositide-dependent phosphorylation. Annu Rev Biochem, 1999. 68: p. 965-1014.
Chang, H. Y. and X. Yang, Proteases for cell suicide: functions and regulation of caspases. Microbiol. Mol Biol Rev, 2000. 64(4): p. 821-46.
Cheng, E. H., et al., BCL-2, BCL-X(L) sequester BH3 domain-only molecules preventing BAX- and BAK-mediated mitochondrial apoptosis. Mol Cell, 2001. 8(3): p. 705-11.
Cheng, J. Q., et al., The Akt/PKB pathway: molecular target for cancer drug discovery. Oncogene, 2005. 24(50): p. 7482-92.
Chipuk, J. E., et al., Direct activation of Bax by p53 mediates mitochondrial membrane permeabilization and apoptosis. Science, 2004. 303(5660): p. 1010-4.
Cross, D. A., et al., Inhibition of glycogen synthase kinase-3 by insulin mediated by protein kinase B. Nature, 1995. 378(6559): p. 785-9.
Dagher, P. C., Apoptosis in ischemic renal injury: roles of GTP depletion and p53. Kidney Int, 2004. 66(2): p. 506-9.
Dai, Y., P. Dent, and S. Grant, Induction of apoptosis in human leukemia cells by the CDK1 inhibitor CGP74514A. Cell Cycle, 2002. 1(2): p. 143-52.
Datta, S. R., A. Brunet, and M. E. Greenberg, Cellular survival: a play in three Akts. Genes Dev, 1999. 13(22): p. 2905-27.
David-Pfeuty, T., Potent inhibitors of cyclin-dependent kinase 2 induce nuclear accumulation of wild-type p53 and nucleolar fragmentation in human untransformed and tumor-derived cells. Oncogene, 1999. 18(52): p. 7409-22.
DeFeo-Jones, D., et al., Tumor cell sensitization to apoptotic stimuli by selective inhibition of specific Akt/PKB family members. Mol Cancer Ther, 2005. 4(2): p. 271-9.
Deveraux, Q. L., et al., X-linked IAP is a direct inhibitor of cell-death proteases. Nature, 1997. 388(6639): p. 300-4.
Dong, J. T., Prevalent mutations in prostate cancer. J Cell Biochem, 2006. 97(3): p. 433-47.
Eaton, S. R., et al., Design of peptidomimetics that inhibit the association of phosphatidylinositol 3-kinase with platelet-derived growth factor-beta receptor and possess cellular activity. J Med Chem, 1998. 41(22): p. 4329-42.
Feldman, B. J. and D. Feldman, The development of androgen-independent prostate cancer. Nat Rev Cancer, 2001. 1(1): p. 34-45.
Feun, L. G., et al., Phase I study of tricyclic nucleoside phosphate using a five-day continuous infusion schedule. Cancer Res, 1984. 44(8): p. 3608-12.
Feun, L. G., et al., A phase II trial of tricyclic nucleoside phosphate in patients with advanced squamous cell carcinoma of the cervix. A Gynecologic Oncology Group Study. Am J Clin Oncol, 1993. 16(6): p. 506-8.
Fischer, P. M. and A. Gianella-Borradori, Recent progress in the discovery and development of cyclin-dependent kinase inhibitors. Expert Opin Investig Drugs, 2005. 14(4): p. 457-77.
Franke, T. F., et al., The protein kinase encoded by the Akt proto-oncogene is a target of the PDGF-activated phosphatidylinositol 3-kinase. Cell, 1995. 81(5): p. 727-36.
Hakem, R., et al., Differential requirement for caspase 9 in apoptotic pathways in vivo. Cell, 1998. 94(3): p. 339-52.
Hahntow, I. N., et al., Cyclin-dependent kinase inhibitor Roscovitine induces apoptosis in chronic lymphocytic leukemia cells. Leukemia, 2004. 18(4): p. 747-55.
Haupt, Y., p53 Regulation: a family affair. Cell Cycle, 2004. 3(7): p. 884-5.
Haupt, Y., et al., p53 mediated apoptosis in HeLa cells: transcription dependent and independent mechanisms. Leukemia, 1997. 11 Suppl 3: p. 337-9.
Holcik, M., H. Gibson, and R. G. Korneluk, XIAP: apoptotic brake and promising therapeutic target. Apoptosis, 2001. 6(4): p. 253-61.
Horoszewicz, J. S., et al., The LNCaP cell line—a new model for studies on human prostatic carcinoma. Prog Clin Biol Res, 1980. 37: p. 115-32.
Kaighn, M. E., et al., Establishment and characterization of a human prostatic carcinoma cell line (PC-3). Invest Urol, 1979. 17(1): p. 16-23.
Kim, E. H., et al., Roscovitine sensitizes glioma cells to TRAIL-mediated apoptosis by downregulation of survivin and XIAP. Oncogene, 2004. 23(2): p. 446-56.
Kotala, V., et al., Potent induction of wild-type p53-dependent transcription in tumour cells by a synthetic inhibitor of cyclin-dependent kinases. Cell Mol Life Sci, 2001. 58(9): p. 1333-9.
Kuida, K., et al., Reduced apoptosis and cytochrome c-mediated caspase activation in mice lacking caspase 9. Cell, 1998. 94(3): p. 325-37.
Kuwana, T., et al., BH3 domains of BH3-only proteins differentially regulate Bax-mediated mitochondrial membrane permeabilization both directly and indirectly. Mol Cell, 2005. 17(4): p. 525-35.
Li, L., et al., The emerging role of the PI3-K-Akt pathway in prostate cancer progression. Prostate Cancer Prostatic Dis, 2005. 8(2): p. 108-18.
Liao, Y., et al., Increase of AKT/PKB expression correlates with gleason pattern in human prostate cancer. Int J Cancer, 2003. 107(4): p. 676-80.
Lin, J., et al., The phosphatidylinositol 3′-kinase pathway is a dominant growth factor-activated cell survival pathway in LNCaP human prostate carcinoma cells. Cancer Res, 1999. 59(12): p. 2891-7.
Lorenzo, E., et al., Doxorubicin induces apoptosis and CD95 gene expression in human primary endothelial cells through a p53-dependent mechanism. J Biol Chem, 2002. 277(13): p. 10883-92.
Lu, W., et al., Activation of p53 by roscovitine-mediated suppression of MDM2 expression. Oncogene, 2001. 20(25): p. 3206-16.
Majumder, P. K. and W. R. Sellers, Akt-regulated pathways in prostate cancer. Oncogene, 2005. 24(50): p. 7465-74.
Martelli, A. M., et al., Phosphoinositide 3-kinase/Akt signaling pathway and its therapeutical implications for human acute myeloid leukemia. Leukemia, 2006. 20(6): p. 911-928.
McClue, S. J., et al., In vitro and in vivo antitumor properties of the cyclin dependent kinase inhibitor CYC202 (R-roscovitine). Int J Cancer, 2002. 102(5): p. 463-8.
Meijer, L., et al., Biochemical and cellular effects of roscovitine, a potent and selective inhibitor of the cyclin-dependent kinases cdc2, cdk2 and cdk5. Eur J Biochem, 1997. 243(1-2): p. 527-36.
Mihara, M., et al., Cyclin-dependent kinase inhibitor (roscovitine) suppresses growth and induces apoptosis by regulating Bcl-x in head and neck squamous cell carcinoma cells. Int J Oncol, 2002. 21(1): p. 95-101.
Mohapatra, S., et al., Roscovitine inhibits STAT5 activity and induces apoptosis in the human leukemia virus type 1-transformed cell line MT-2. Cancer Res, 2003. 63(23): p. 8523-30.
Mohapatra, S., et al., Accumulation of p53 and reductions in XIAP abundance promote the apoptosis of prostate cancer cells. Cancer Res, 2005. 65(17): p. 7717-23.
Mohapatra, S., et al., Roscovitine inhibits differentiation and invasion in a three-dimensional skin reconstruction model of metastatic melanoma. Mol Cancer Res, 2007. 5(2): p. 145-51.
Moll, U. M. and A. Zaika, Nuclear and mitochondrial apoptotic pathways of p53. FEBS Lett, 2001. 493(2-3): p. 65-9.
Murillo, H., et al., Role of PI3K signaling in survival and progression of LNCaP prostate cancer cells to the androgen refractory state. Endocrinology, 2001. 142(11): p. 4795-805.
Nutley, B. P., et al., Metabolism and pharmacokinetics of the cyclin-dependent kinase inhibitor R-roscovitine in the mouse. Mol Cancer Ther, 2005. 4(1): p. 125-39.
Patterson, S. G., et al., Novel role of Stat1 in the development of docetaxel resistance in prostate tumor cells. Oncogene, 2006. 25(45): p. 6113-22.
Pettaway, C. A., et al., Selection of highly metastatic variants of different human prostatic carcinomas using orthotopic implantation in nude mice. Clin Cancer Res, 1996. 2(9): p. 1627-36.
Raje, N., et al., Seliciclib (CYC202 or R-roscovitine), a small-molecule cyclin-dependent kinase inhibitor, mediates activity via down-regulation of Mcl-1 in multiple myeloma. Blood, 2005. 106(3): p. 1042-7.
Raynaud, F. I., et al., Cassette dosing pharmacokinetics of a library of 2,6,9-trisubstituted purine cyclin-dependent kinase 2 inhibitors prepared by parallel synthesis. Mol Cancer Ther, 2004. 3(3): p. 353-62.
Raynaud, F. I., et al., In vitro and in vivo pharmacokinetic-pharmacodynamic relationships for the trisubstituted aminopurine cyclin-dependent kinase inhibitors olomoucine, bohemine and CYC202. Clin Cancer Res, 2005. 11(13): p. 4875-87.Senderowicz, A. M., Small-molecule cyclin-dependent kinase modulators. Oncogene, 2003. 22(42): p. 6609-20.
Senderowicz, A. M., Novel small molecule cyclin-dependent kinases modulators in human clinical trials. Cancer Biol Ther, 2003. 2(4 Suppl 1): p. S84-95.
Shapiro, G. I., Cyclin-dependent kinase pathways as targets for cancer treatment. J Clin Oncol, 2006. 24(11): p. 1770-83.Srinivasula, S. M., et al., A conserved XIAP-interaction motif in caspase-9 and Smac/DIABLO regulates caspase activity and apoptosis. Nature, 2001. 410(6824): p. 112-6.
Stocker, H., et al., Living with lethal PIP3 levels: viability of flies lacking PTEN restored by a PH domain mutation in Akt/PKB. Science, 2002. 295(5562): p. 2088-91.
Strasser, A., L. O'Connor, and V. M. Dixit, Apoptosis signaling. Annu Rev Biochem, 2000. 69: p. 217-45.
Thomas, D. J., et al., p53 expression and clinical outcome in prostate cancer. Br J Urol, 1993. 72(5 Pt 2): p. 778-81.
Tirado, O. M., S. Mateo-Lozano, and V. Notario, Roscovitine is an effective inducer of apoptosis of Ewing's sarcoma family tumor cells in vitro and in vivo. Cancer Res, 2005. 65(20): p. 9320-7.
van Bokhoven, A., et al., Molecular characterization of human prostate carcinoma cell lines. Prostate, 2003. 57(3): p. 205-25.
Vanhaesebroeck, B. and D. R. Alessi, The PI3K-PDK1 connection: more than just a road to PKB. Biochem J, 2000. 346 Pt 3: p. 561-76.
Vaux, D. L. and J. Silke, Mammalian mitochondrial IAP binding proteins. Biochem Biophys Res Commun, 2003. 304(3): p. 499-504.
Vogelstein, B., D. Lane, and A. J. Levine, Surfing the p53 network. Nature, 2000. 408(6810): p. 307-10.
Wang, D., et al., Inhibition of human immunodeficiency virus type 1 transcription by chemical cyclin-dependent kinase inhibitors. J Virol, 2001. 75(16): p. 7266-79.
Wei, M. C., et al., Proapoptotic BAX and BAK: a requisite gateway to mitochondrial dysfunction and death. Science, 2001. 292(5517): p. 727-30.
Wojciechowski, J., et al., Rapid onset of nucleolar disintegration preceding cell cycle arrest in roscovitine-induced apoptosis of human MCF-7 breast cancer cells. Int J Cancer, 2003. 106(4): p. 486-95.
Wotring, L. L., et al., Dual mechanisms of inhibition of DNA synthesis by triciribine. Cancer Res, 1990. 50(16): p. 4891-9.
Yamaguchi, H. and H. G. Wang, Bcl-XL protects BimEL-induced Bax conformational change and cytochrome C release independent of interacting with Bax or BimEL. J Biol Chem, 2002. 277(44): p. 41604-12.
Yang, E., et al., Bad, a heterodimeric partner for Bcl-XL and Bcl-2, displaces Bax and promotes cell death. Cell, 1995. 80(2): p. 285-91.
Yang, L., et al., Akt/protein kinase B signaling inhibitor-2, a selective small molecule inhibitor of Akt signaling with antitumor activity in cancer cells overexpressing Akt. Cancer Res, 2004. 64(13): p. 4394-9.
Yuan, X. J. and Y. E. Whang, PTEN sensitizes prostate cancer cells to death receptor-mediated and drug-induced apoptosis through a FADD-dependent pathway. Oncogene, 2002. 21(2): p. 319-27.
Zegarra-Moro, O. L., et al., Disruption of androgen receptor function inhibits proliferation of androgen-refractory prostate cancer cells. Cancer Res, 2002. 62(4): p. 1008-13.
Zong, W. X., et al., BH3-only proteins that bind pro-survival Bcl-2 family members fail to induce apoptosis in the absence of Bax and Bak. Genes Dev, 2001. 15(12): p. 1

The disclosure of all publications cited above are expressly incorporated herein by reference, each in its entirety, to the same extent as if each were incorporated by reference individually.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall therebetween. Now that the invention has been described,

What is claimed is:

1. A combination therapy for the treatment of cancer comprising a therapeutically effective amount of a Cdk9 inhibitors and one or more PI3K/Akt inhibitors.

2. The combination therapy according to claim 1 wherein one of the one or more PI3K/Akt inhibitors is selected from the group consisting of API-2 (Triciribine) and LY294002.

3. The combination therapy according to claim 1 wherein the Cdk 9 inhibitor is selected from the group consisting of roscovitine and flavopiridol.

4. A combination therapy for the treatment of cancer comprising a therapeutically effective amount of roscovitine and one or more PI3K/Akt inhibitors.

5. The combination therapy according to claim 4 wherein one of the one or more PI3K/Akt inhibitors is selected from the group consisting of API-2 and LY (LY294002).

6. A method of treating prostate cancer in a subject comprising the step of administering roscovitine and one or more PI3K/Akt inhibitors in a therapeutically effective amount to a subject in need thereof.

7. The method according to claim 6 further comprising the step of performing androgen ablation therapy.

8. The method according to claim 6 wherein the prostate cancer is androgen-independent prostate cancer.

9. The method according to claim 6 wherein the one or more Akt inhibitors is selected from the group consisting of API-2 and LY (LY294002).

10. A method of treating prostate cancer in a subject comprising the step of administering in combination API-2 and one or more inhibitors of XIAP in a therapeutically effective amount to a subject in need thereof.

11. The method according to claim 10 wherein the XIAP inhibitor depletes Cdk-9.

12. The method according to claim 10 wherein the XIAP inhibitor is roscovitine.

13. The method according to claim 12 wherein the prostate cancer is androgen-independent prostate cancer.

14. A method of treating cancer in a subject comprising the step of administering roscovitine and one or more PI3K/Akt inhibitors in a therapeutically effective amount to a subject in need thereof.

15. The method according to claim 14 wherein one of the one or more PI3K/AKT inhibitors is selected from the group consisting of API-2 and LY294002.

16. The method according to claim 14 wherein the cancer is selected from the group consisting of prostate cancer, sarcoma and mantle cell lymphoma.

17. A method for treating a tumor or cancer in a mammal comprising (i) obtaining a biological sample from the tumor or cancer; (ii) determining whether the tumor or cancer overexpresses an Akt kinase, (iii) if the tumor or cancer overexpresses Akt kinase, treating the tumor or cancer with an effective amount of a combination therapy comprising API-2 and roscovitine.

18. The method of claim 17 wherein the level of Akt kinase expression is determined by assaying the cancer for the presence of a phosphorylated Akt kinase.

19. The method according to claim 17 wherein the mammal is a human.

20. The method according to claim 17 wherein the cancer is prostate cancer.

21. The method according to claim 20 wherein the prostate cancer is androgen-independent prostate cancer.

22. The method according to claim 20 further comprising the step of performing androgen ablation therapy.

23. The method according to claim 17 further comprising the step of determining whether the cancer expresses p53, wherein the expression of wt p53 correlates favorably with responsiveness to treatment with the combination therapy API-2 (Triciribine) and roscovitine.
